(19) United States
(12) Patent Application Publication
(10) Pub. No.: US 2024/0148456 A1
(43) Pub. Date: May 9, 2024

(54) TRANSMISSION AND CONNECTION STRUCTURE FOR CONNECTING INSTRUMENT DRIVE AND STERILE ADAPTER, AND SURGICAL ROBOT INCLUDING THE SAME

(71) Applicant: CORNERSTONE TECHNOLOGY (SHENZHEN) LIMITED, Shenzhen (CN)

(72) Inventor: Qiusheng YANG, Shenzhen (CN)

(21) Appl. No.: 18/412,470

(22) Filed: Jan. 13, 2024

Related U.S. Application Data
(63) Continuation of application No. PCT/CN2022/100523, filed on Jun. 22, 2022.

(30) Foreign Application Priority Data
Jul. 14, 2021 (CN) 202110795340.8

(51) Int. Cl. A61B 34/37; A61B 17/00; A61B 46/10; A61B 34/30

(52) U.S. Cl. CPC A61B 34/37; A61B 17/00; A61B 46/10; A61B 2017/00477; A61B 2034/301

(57) ABSTRACT

The present disclosure discloses a transmission and connection structure for connecting an instrument drive and a sterile adapter, and a surgical robot. The instrument drive includes a transmission and connection member for the instrument drive. The transmission and connection member for the instrument drive has a first connection end configured to be coupled to the sterile adapter, and the first connection end is provided with a first toothed portion. The sterile adapter includes a transmission and connection member for the sterile adapter, the transmission and connection member for the sterile adapter has a second connection end configured to be coupled to the first connection end of the instrument drive, and the second connection end is provided with a second toothed portion. The second toothed portion is configured to engage with the first toothed portion.

### CROSS-REFERENCE TO RELATED APPLICATION

The present application is a continuation of PCT Patent Application No. PCT/CN2022/100523, entitled “TRANSMISSION CONNECTION STRUCTURE OF INSTRUMENT DRIVE AND STERILE ADAPTER, AND SURGICAL ROBOT,” filed on Jun. 22, 2022, which claims priority to Chinese patent application No. 202110795340.8, entitled “TRANSMISSION CONNECTION STRUCTURE OF INSTRUMENT DRIVER AND STERILE ADAPTER, AND SURGICAL ROBOT,” filed Jul. 14, 2021, each of which is incorporated by reference herein in its entirety.

### TECHNICAL FIELD

The present disclosure relates to the field of medical device technology, specifically to a transmission and connection structure for connecting an instrument drive and a sterile adapter, and a surgical robot including the same.

### BACKGROUND

A surgical robot can help a doctor achieve precise positioning in surgeries, with the advantages of reducing patient wounds and shortening postoperative recovery time. And the surgical robot has a stable operating platform that can solve problems caused by trembling and other situations of the doctor, so that the surgical robot is widely applied in clinical surgical operations. The surgical robot on a patient side is configured to perform surgical operations using surgical tools with at least one end effector. To meet the usage needs of different surgical instruments during surgery, surgical instruments and instrument drives are generally designed to be detachable for replacement of different surgical instruments during surgery. In addition, surgical instruments are generally capable of independent disinfection and sterilization. An instrument drive end is generally designed as non sterile. To ensure sterility during the surgical operations, a sterile adapter needs to be added between the instrument drive and the surgical instrument during surgery to isolate the non sterile instrument drive end and the sterile surgical instrument end during surgery.

In related technologies, the transmission and connection between the instrument drive and the sterile adapters mostly adopts a single point connection method, which requires a single protrusion on a transmission and connection member for the instrument drive to be orientated in the same direction as an orientation of a single recess on a transmission and connection member for the sterile adapter, and the protrusion and the recess is in one-to-one correspondence. The single point connection method requires a lot of actions during operation, and a certain gap between the protrusions and the recess during the matching to ensure assembly reliability. However, the gap can affect torque transmission and generate transmission clearance, resulting in a relatively low reliability.

### SUMMARY

In a first aspect, a transmission and connection structure for connecting an instrument drive and a sterile adapter is provided according to the embodiments of the present disclosure. The instrument drive includes a transmission and connection member for the instrument drive. The transmission and connection member for the instrument drive has a first connection end configured to be coupled to the sterile adapter, and the first connection end is provided with a first toothed portion. The sterile adapter includes a transmission and connection member for the sterile adapter, the transmission and connection member for the sterile adapter has a second connection end configured to be coupled to the first connection end of the instrument drive, and the second connection end is provided with a second toothed portion. The second toothed portion is configured to engage with the first toothed portion.

According to the transmission and connection structure for connecting the instrument drive and the sterile adapter of the present disclosure, the meshing of the toothed structure enables the transmission and connection member for the sterile adapter and the transmission and connection member for the instrument drive to achieve any connection in the entire circumferential direction, and the meshing connection is tight, and occurrence of gaps is reduced, thereby improving the stability and accuracy of the transmission.

In a second aspect, a surgical robot is further provided according to the embodiments of the present disclosure, and the surgical robot includes a transmission and connection structure for connecting the instrument drive and sterile adapter in the first aspect mentioned above.

According to the present disclosure, the surgical robot can achieve a technical effect similar to the transmission and connection structure for connecting the instrument drive and sterile adapter in the first aspect mentioned above.

### BRIEF DESCRIPTION OF THE DRAWINGS

The following drawings of the present disclosure are herein used as a part of the present disclosure for understanding the present disclosure. Embodiments of the present disclosure are described with reference to the accompanying drawings to explain the principles of the present disclosure, in which:

FIG. 1 is a partial three-dimensional schematic view of a surgical robot provided according to a first optional embodiment of the present disclosure;

FIG. 2 is an explosive view of the surgical robot shown in FIG. 1;

FIG. 3 is an explosive view of the surgical robot shown in FIG. 2 from another perspective;

FIG. 4 is a three-dimensional schematic view of a transmission and connection member for the instrument drive shown in FIG. 2;

FIG. 5 is a three-dimensional schematic view of the transmission and connection member for the instrument drive in FIG. 4 from another perspective;

FIG. 6 is a cross-sectional view of the transmission and connection member for the instrument drive shown in FIG. 4 along A-A′;

FIG. 7 is a cross-sectional view of the transmission and connection member for the instrument drive shown in FIG. 4 along B-B′;

FIG. 8 is a three-dimensional view of the transmission and connection member for the sterile adapter shown in FIG. 2;

FIG. 9 is a cross-sectional view of the transmission and connection member for the sterile adapter shown in FIG. 8 along C-C′;

FIG. 10 is a cross-sectional view of the transmission and connection member for the sterile adapter shown in FIG. 8 along D-D′;

FIG. 11 is a partial three-dimensional schematic view of a surgical robot provided according to a second optional embodiment of the present disclosure;

FIG. 12 is a three-dimensional view of the transmission and connection member for the instrument drive shown in FIG. 11;

FIG. 13 is a front view of the transmission and connection member for the instrument drive shown in FIG. 12;

FIG. 14 is a three-dimensional view of the transmission and connection member for the sterile adapter shown in FIG. 11;

FIG. 15 is a three-dimensional view of the transmission and connection member for the sterile adapter shown in FIG. 14 from another perspective;

FIG. 16 is a front view of the transmission and connection member for the sterile adapter shown in FIG. 14; and

FIG. 17 is a cross-sectional view of the transmission and connection member for the sterile adapter shown in FIG. 14.

### DETAILED DESCRIPTION OF THE EMBODIMENTS

Detailed illustration is given in the following description to provide a more thorough understanding of the present disclosure. However, it will be apparent to those skilled in the art that embodiments of the present disclosure may be practiced without one or more of these details. In other examples, some technical features well known in the art are not described in order to avoid confusion with embodiments of the present disclosure.

The terms referenced in the present disclosure are only for describing specific embodiments, and are not intended to limit the exemplary embodiments according to the present disclosure. As used here, unless the context otherwise clearly indicates, the singular form may also intended to include the plural form. In addition, in response to the terms “comprising” and/or “including” being referenced in the present disclosure, they indicate the existence of the said features, entirety, step, operation, element, and/or component, but do not exclude the existence or addition of one or more other features, entirety, step, operation, element, component, and/or their combination. Ordinal numbers such as “first” and “second” referenced in the present disclosure are merely identifiers and do not have any other meaning, e.g., does not represent a specific order. In addition, for example, the term “first component/member” does not imply the existence of a “second component/member”, and the term “second component/member” does not imply the existence of the “first component/member”.

The terms “up”, “down”, “front”, “rear”, “left”, “right”, and similar expressions referenced in the present disclosure are merely intended to explain the present disclosure rather than limit the present disclosure.

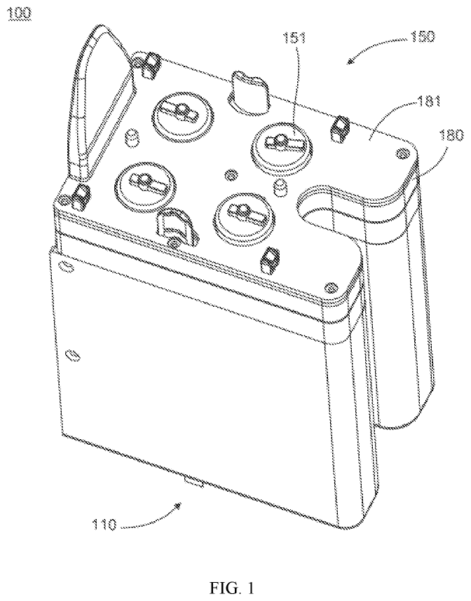

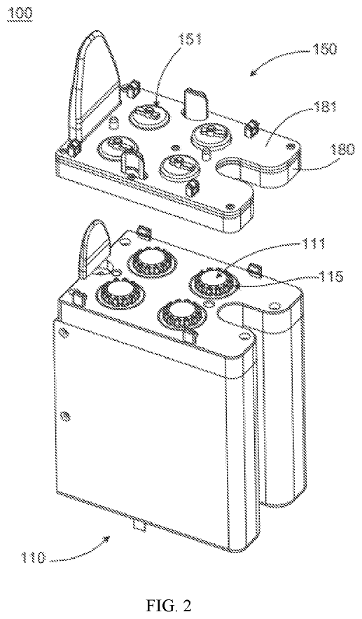

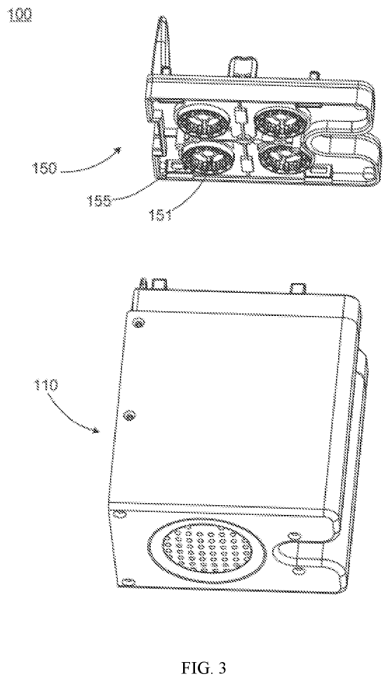

According to the first optional embodiment of the present disclosure, referring first to FIG. 1, FIG. 2, and FIG. 3, a surgical robot 100 includes a surgical instrument (not shown), a sterile adapter 150, and an instrument drive 110. The sterile adapter 150 is connected to the instrument drive 110, and the surgical instrument is connected to the sterile adapter 150. Specifically, a lower surface of the sterile adapter 150 is connected to an upper surface of the instrument drive 110. A front-end of the surgical instrument is configured as surgical tools such as forceps, scissors, clamps, etc. A rear end of the surgical instrument is connected to an upper surface of the sterile adapter 150, and the instrument drive 110 is configured to provide drive force to the instrument executing mechanism in the middle of the rear end of the surgical instrument via the sterile adapter 150, so that the pulling components (e.g., wires or ropes) in a shaft enable the above surgical tools to complete movement such as pitching, deflection, and gripping. A surface of instrument drive 110 is engaged with the lower surface of sterile adapter 150. A rear end surface of the surgical instrument is engaged with the upper surface of sterile adapter 150. During operation, the surface of instrument drive 110, the lower surface of sterile adapter 150, the upper surface of sterile adapter 150, and the rear end surface of the surgical instrument do not change in relative position. The transmission and connection member 111 for the instrument drive 110, the transmission and connection member 151 for the sterile adapter 150, and a transmission and connection member for the surgical instrument at the rear end are connected and fixed in specific ways, respectively, so that during operation, the instrument drive 110 is configured to drive the transmission and connection member 111 for the instrument drive 110 to rotate and transmit the driving force (or torque) to the transmission and connection member for the surgical instrument at the rear end via the transmission and connection member 151 for the sterile adapter 150, thereby achieving control of the surgical tools. The transmission and connection structure for connecting the instrument drive and the sterile adapter is described in this embodiment.

The instrument drive 110 includes a transmission and connection member 111 for the instrument drive 110, the sterile adapter 150 includes a transmission and connection member 151 for the sterile adapter 150, the transmission and connection member 151 for the sterile adapter 150 is connected to the transmission and connection member 111 for the instrument drive 110, and the transmission and connection member for the surgical instrument at the rear end is connected to the transmission and connection member 151 for the sterile adapter 150.

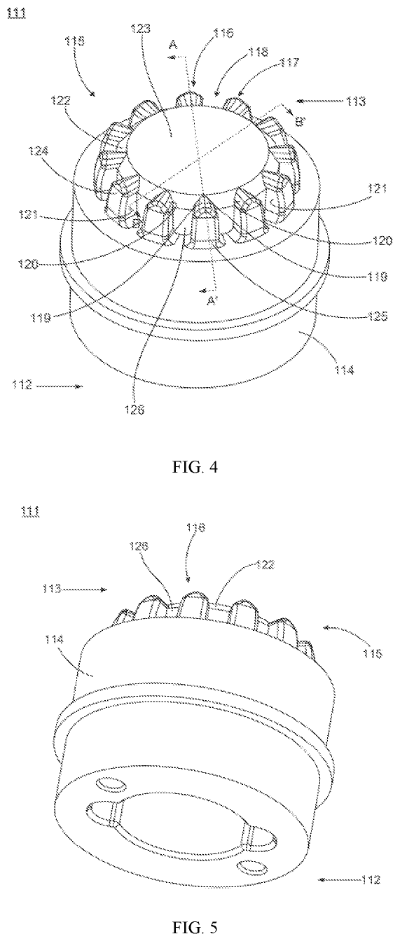

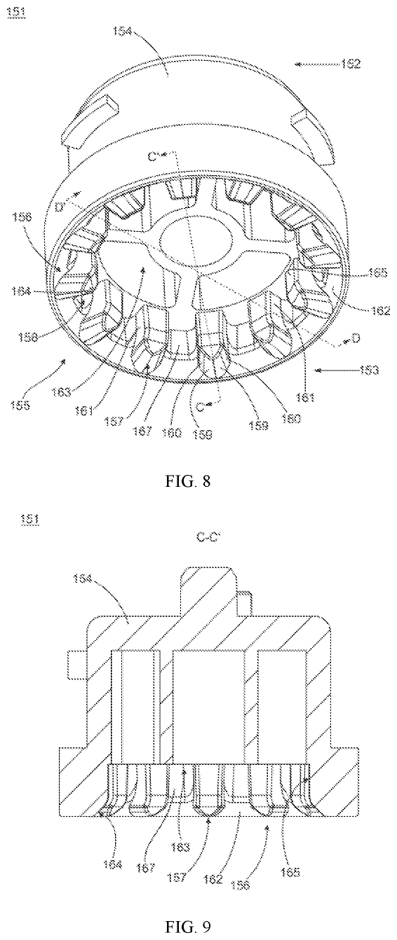

Specifically, referring to FIG. 2, FIG. 3, FIG. 4, FIG. 5, and FIG. 8, the transmission and connection member 111 for the instrument drive 110 has a first drive end 112 and a first connection end 113. The first drive end 112 is arranged away from the sterile adapter 150 and is configured to connect to a drive component (not shown, may be a motor) inside the instrument drive 110. The first connection end 113 is close to the sterile adapter 150 and is configured to be coupled with the transmission and connection member 151 for the sterile adapter 150.

The transmission and connection member 151 for the sterile adapter 150 has a second drive end 152 and a second connection end 153. The second connection end 153 is close to the instrument drive 110 and is configured to be coupled with the transmission and connection member 111 for the instrument drive 110. The second drive end 152 is arranged away from the instrument drive 110 and is configured to be coupled with the transmission and connection member for the surgical instrument at the rear end. The transmission and connection member 111 for the instrument drive 110 further has a first toothed portion 115 arranged at the first connection end 113. The transmission and connection member 151 for the sterile adapter 150 further has a second toothed portion 155 arranged at the second connection end 153. Moreover, the second toothed portion 155 is configured to engage with the first toothed portion 115.

Therefore, according to the surgical instrument of the present disclosure, the meshing of two toothed structures can achieve connection between the transmission and connection member 151 for the sterile adapter 150 and the transmission and connection member 111 for the instrument drive 110 at any point around the entire circumference, and the meshing connection is tight without gaps, thereby improving the stability and accuracy of the transmission.

The following will provide a detailed explanation of the transmission and connection member 111 for the instrument drive 110 in conjunction with FIG. 4 to FIG. 7. The transmission and connection member 111 for the instrument drive 110 has a first body portion 114 and a positioning portion protruding from the first body portion 114. Therefore, in this embodiment, the positioning portion is configured as a protruding portion 123. The end of the positioning portion forms the first connection end 113. The first toothed portion 115 is arranged at the top of the first body portion 114 and along the circumference of the first body portion 114. Specifically, the first toothed portion 115 is arranged around the protruding portion 123. The first body portion 114 and the positioning portion may both be optionally configured as a cylindrical shape.

The first toothed portion 115 includes multiple first teeth 116, which can be odd or even in number. The multiple first teeth 116 are uniformly arranged along the circumference of the transmission and connection member 111 for the instrument drive 110, and each of the multiple first teeth 116 is connected to the protruding portion 123.

Each of the multiple first teeth 116 has the same structure, and each of the multiple first teeth 116 is symmetrical relative to the axial section of the first body portion 114. That is, each of the multiple first teeth 116 is symmetrical about its own central plane as the plane of symmetry, which is conducive to direct installation, thereby making installation convenient and reducing the alignment operations between the transmission and connection member 111 for the instrument drive 110 and the transmission and connection member 151 for the sterile adapter 150.

Each of the multiple first teeth 116 has a first tooth peak 117, and a first tooth trough 118 is formed between two adjacent first teeth 116. On both sides of each of the multiple first teeth 116, a pair of symmetrical first guide surfaces 119, a pair of symmetrical first transition surfaces 120, and a pair of symmetrical first engaging surfaces 121 are sequentially arranged along the direction from the first tooth peak 117 to the root of the respective one of the multiple first teeth 116.

The pair of first guide surfaces 119 meet at the first tooth peak 117, or specifically, each first guide surface 119 is inclined towards the first tooth peak 117, and is formed as a flat surface, which has an inclined angle, or the first guide surface 119 is formed as a smoothly curved surface, so that the first tooth peak 117 is formed as a sharp corner or a rounded corner. Therefore, the radial cross-sectional area (or radial cross-sectional width) of each of the multiple first teeth 116 at the first tooth peak 117 is smaller than the radial cross-sectional area (or radial cross-sectional width) of each of the multiple first teeth 116 at the root of the respective one of the multiple first teeth 116, resulting in each of the multiple first teeth 116 forming a shape with one end wide and one end narrow.

The pair of first engaging surfaces 121 extend along a height direction of each of the multiple first teeth 116 to the first body portion 114. The pair of first engaging surfaces 121 are formed as vertical surfaces or slightly inclined surfaces. The first engaging surface 121 is configured to be in contact and mesh with the second tooth 156 to transmit torque. The first engaging surface 121 can transmit torque to the maximum extent when formed as a vertical surface. The first engaging surface 121 may also be formed as a slightly inclined surface, which can seek a balance between transmitting torque and accuracy requirements. In addition, the slightly inclined structure of the first engaging surface 121 can achieve a highly accurate fit when matched with the corresponding matching surface of the each of the multiple second teeth 116. Specifically, a slightly inclined surface can refer to a surface inclined by an angle within 15 degrees from the vertical direction. Optionally, the included angle between the slightly inclined surface and the vertical direction is less than or equal to 5 degrees.

The first transition surface 120 is connected between the first guide surface 119 and the first engaging surface 121 to serve as a transition therebetween and perform guiding. The inclined degree of the first transition surface 120 is greater than that of the first guide surface 119, thereby achieving a gentle transition from the first guide surface 119 to the first engaging surface 121.

The protruding portion 123 includes a first matching inclined surface 122 and a first matching cylindrical surface 126 both extending along the circumference of the protruding portion 123. The first matching inclined surface 122 is formed at the top portion of the protruding portion 123 and extends along a circumference of the protruding portion 123, and a radial cross-sectional diameter of the first matching inclined surface 122 gradually increases along a direction from the first tooth peak 117 to the root of the respective one of the multiple first teeth 116. In other words, the first matching inclined surface 122 can be formed as a straight inclined surface or an arc inclined surface, such as a rounded corner or a chamfer. The first matching cylindrical surface 126 is adjacent to the first matching inclined surface 122. The first matching cylindrical surface 126 and the first matching inclined surface 122 mentioned above can be formed as connecting surfaces between the two first teeth 116 on the protruding portion 123.

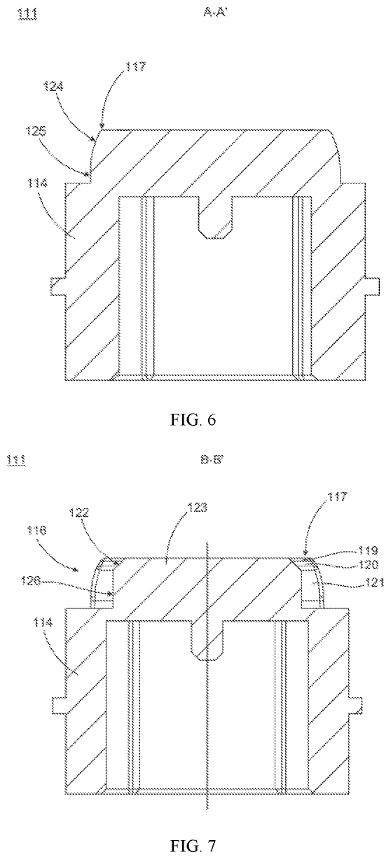

Referring to FIG. 4, FIG. 5, and FIG. 6, an outer circumferential surface of the first toothed portion 115 arranged at an outer circumferential side of each of the multiple first teeth 116 transits from the first smooth arc surface 124 to a first cylindrical surface 125 along the direction from the first tooth peak 117 to the root of the respective one of the multiple first teeth 116. The first smooth arc surface 124 gradually increases in radial cross-sectional radius or radial cross-sectional diameter along a direction from the first tooth peak 117 to the first cylindrical surface 125. Therefore, the first smooth arc surface 124 substantially forms a shape inclined towards the middle of the transmission and connection member 111 for the instrument drive 110. Specifically, the radial cross-sectional radius or radial cross-sectional diameter of the circumference of the first smooth arc surface 124 gradually increases from the first tooth peak 117 to the root of the respective one of the multiple first teeth 116. The first cylindrical surface 125 extends from the first smooth arc surface 124 to the first body portion 114.

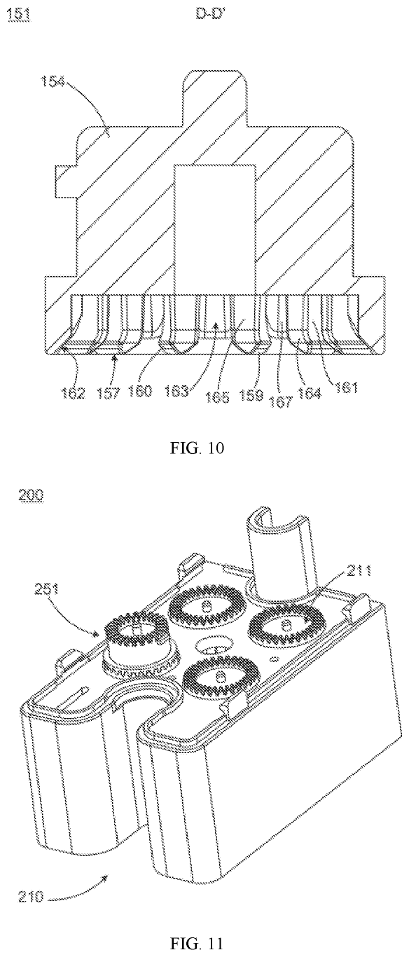

The transmission and connection member 151 for the sterile adapter 150 will be described in detail below in conjunction with FIG. 8, FIG. 9, and FIG. 10. The transmission and connection member 151 for the sterile adapter 150 has a second body portion 154, which is recessed inward at the second connection end 153 to form a matching portion. The matching portion shown in the figure is in the shape of a cover and can be engaged with the first connection end. Therefore, in this embodiment, the matching portion may also be referred to as the recessing portion 163. The recessing portion 163 is opened at the second connection end 153, and the recessing portion 163 is configured to accommodate the protruding portion 123 of the transmission and connection member 111 for the instrument drive 110, so that the protruding portion 123 can at least partially extend into the recessing portion 163.

The second toothed portion 155 is arranged at the bottom of the second body portion 154 and is arranged along the circumference of the second body portion 154. Specifically, the second toothed portion 155 is arranged within the recessing portion 163 and is located at the opening of the recessing portion 163. The second toothed portion 155 includes multiple second teeth 156 uniformly arranged along the circumference of the transmission and connection member 151 for the sterile adapter 150. Each of the multiple second teeth 156 has the same structure, and is symmetrical relative to the axial section of the second body portion 154.

Each of the multiple second teeth 116 has a second tooth peak 157, and a second tooth trough 158 is formed between two adjacent second teeth 156. In engagement of the first toothed portion 115 with the second toothed portion 155, each of the multiple first teeth 116 extends into the second toothed trough 158, and the each of the multiple second teeth 116 extends into the first toothed trough 118. In other words, in response to the transmission and connection member 111 for the instrument drive 110 being connected to the transmission and connection member 151 for the sterile adapter 150, the protruding portion 123 is matched with the recessing portion 163, and the first toothed portion 115 is matched with the second toothed portion 155, so that the transmission and connection member 111 for the instrument drive 110 and the transmission and connection member 151 for the sterile adapter 150 can be tightly connected at any position.

The each of the multiple second teeth 116 includes a second guide surface 159 corresponding to the first guide surface 119, a second transition surface 160 corresponding to the first transition surface 120, a second engaging surface 161 corresponding to the first engaging surface 121, a second smooth arc surface 164 similar to the first smooth arc 124, and a second cylindrical surface 165 similar to the first cylindrical surface 125.

Specifically, a pair of second guide surfaces 159 are engaged at the second tooth peak 157, or, the second guide surface 159 is inclined towards the second tooth peak 157, and is formed as a straight surface (straight inclined surface) or a smooth arc surface, so that the second tooth peak 157 is formed as a sharp corner or a rounded corner.

Therefore, the radial cross-sectional area of the each of the multiple second teeth 116 at the second tooth peak 157 is smaller than the radial cross-sectional area of the each of the multiple second teeth 116 at the root of the respective one of the multiple second teeth 116, resulting in the each of the multiple second teeth 116 forming a shape with one end wide and one end narrow. Moreover, both the first tooth peak 117 and the second tooth peak 157 are formed with sharp corners or rounded corners to have a shape with one end wide and one end narrow, which can avoid the first tooth peak 117 and the second tooth peak 157 from being in contact with each other during docking, thereby allowing the first tooth peak 117 to be quickly stagger with the second tooth peak 157 and facilitating rapid installation.

A pair of second engaging surfaces 161 extends along the height direction of the each of the multiple second teeth 116 to the second body portion 154, and the pair of second engaging surfaces 161 are formed as vertical surfaces or slightly inclined surfaces. The second engaging surface 161 is configured to be in contact and mesh with the first engaging surface 121 of each of the multiple first teeth 116.

The second transition surface 160 is connected between the second guide surface 159 and the second engaging surface 161 to serve as a transition therebetween and perform guiding. The inclined degree of the second transition surface 160 is greater than that of the second guide surface 159, thereby achieving a gentle transition from the second guide surface 159 to the second engaging surface 161.

A second matching inclined surface 162 and a second matching cylindrical surface 167 are arranged on the inner wall of the recessing portion 163. The second matching inclined surface 162 is arranged at the circumference of the recessing portion 163 at the second connection end 153, and a radial cross-sectional diameter of the second matching inclined surface 162 gradually decreases along the direction from the second tooth peak 157 to the root of the respective one of the multiple second teeth 116. In other words, the second matching inclined surface 162 can be optionally formed as a straight inclined surface or an arc inclined surface, such as a rounded corner or a chamfer. The second matching cylindrical surface 167 is adjacent to the second matching inclined surface 162. The second matching cylindrical surface 167 and the second matching inclined surface 162 mentioned above can be formed as a connecting surface between the two adjacent second teeth 156 on the inner wall of the recessing portion 163.

A second smooth arc surface 164 and a second cylindrical surface 165 are sequentially arranged from top to bottom on the inner side along the circumference of the each of the multiple second teeth 116. The second smooth arc surface 164 is formed as a smooth arc surface and extends to the second tooth peak 157, thereby connecting with a pair of second guide surfaces 159 and a pair of second transition surfaces 160. As a result, the second smooth arc surface 164 substantially forms a shape inclined towards the outer circumference of the transmission and connection member 151 for the sterile adapter 150. Specifically, the radial cross-sectional radius or radial cross-sectional diameter of the circumference of the second smooth arc surface 164 gradually increases along the direction from the second tooth peak 157 to the root of the respective one of the multiple second teeth 116. The second cylindrical surface 165 extends from the second smooth arc surface 164 to the second body portion 154.

Therefore, in response to the transmission and connection member 111 for the instrument drive 110 being connected to the transmission and connection member 151 for the sterile adapter 150, the first smooth arc surface 124 can be guided in conjunction with the second matching inclined surface 162, and the second smooth arc surface 164 can be guided in conjunction with the first matching inclined surface 122, which makes it easier and smoother for the protruding portion 123 to enter the recessing portion 163. Moreover, the first guide surface 119 can be guided in conjunction with the second guide surface 159, which makes it easier for each of the multiple first teeth 116 to enter the second tooth trough 158 and the each of the multiple second teeth 116 to enter the first tooth trough 118. Thus, the first cylindrical surface 125 of each of the multiple first teeth 116 can form an assembly fit (or mesh) with the second matching cylindrical surface 167, and the second cylindrical surface 165 of the each of the multiple second teeth 116 can form an assembly fit (or mesh) with the first matching cylindrical surface 126.

According to the second optional embodiment of the present disclosure, referring to FIGS. 11 to 17, a main structure of an instrument drive 210 and a sterile adapter (not shown) of the second optional embodiment is the same as that of the first optional embodiment, and a main difference is in the transmission and connection member 211 for the instrument drive 210 and the transmission and connection member 251 for the sterile adapter.

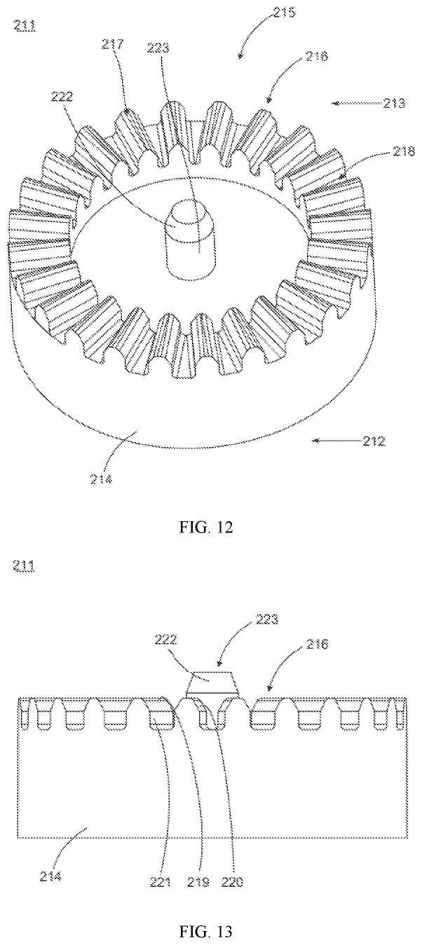

Specifically, FIG. 12 and FIG. 13 illustrate the transmission and connection member 211 for the instrument drive 210 of the second optional embodiment of the present disclosure. The transmission and connection member 211 for the instrument drive 210 has a first drive end 212 and a first connection end 213, which are similar to the first optional embodiment and will not be further described here.

The transmission and connection member 211 for the instrument drive 210 further includes a first body portion 214 and a first toothed portion 215 arranged at the edge of the first body portion 214 along a circumferential direction, and the first toothed portion 215 is continuous in a circumferential direction and is located at the first connection end 213. The structure of each of the multiple first teeth 216 may be identical and symmetrical (axial symmetrical).

The first tooth 216 further includes a first tooth peak 217, a first tooth trough 218, a pair of first guide surfaces 219, a pair of first transition surfaces 220, and a pair of first engaging surfaces 221 similar to the first tooth peak 117, the first tooth trough 118, the pair of first guide surfaces 119, the pair of first transition surfaces 120, and the pair of first engaging surfaces 121 in the first optional embodiment. The difference between the first tooth 216 and the first tooth 116 in the first optional embodiment is that there is no matching surfaces in the outer circumference of the first tooth 216, and assembly is only achieved by relying on the engaging surfaces. The first tooth 216 is formed to extend inward from the circumference of the first body portion 214.

The transmission and connection member 211 for the instrument drive 210 further includes a positioning portion configured as a positioning column 223, which protrudes from the middle of the first body portion 214 at the first connection end 113. In addition, the multiple first teeth 216 are not arranged on the positioning column 223 but are spaced from the positioning column 223. A first matching inclined surface 222 is formed on a top portion of the positioning column 223 along the circumference of the positioning column 223.

In addition, the width of each of the multiple first tooth 216 along the circumferential direction gradually decreases in a radial direction towards the positioning column 223, which can easily cause a partial top surface to be formed on the first tooth peak 217. For example, the outer circumferential side of the first tooth peak 217 shown in FIG. 12 has a top surface, and a sharp corner or a rounded corner is formed on the inner outer circumferential side of the first tooth peak 217. Optionally, the first tooth peak 217 is integrally formed with the sharp corner or the rounded corner.

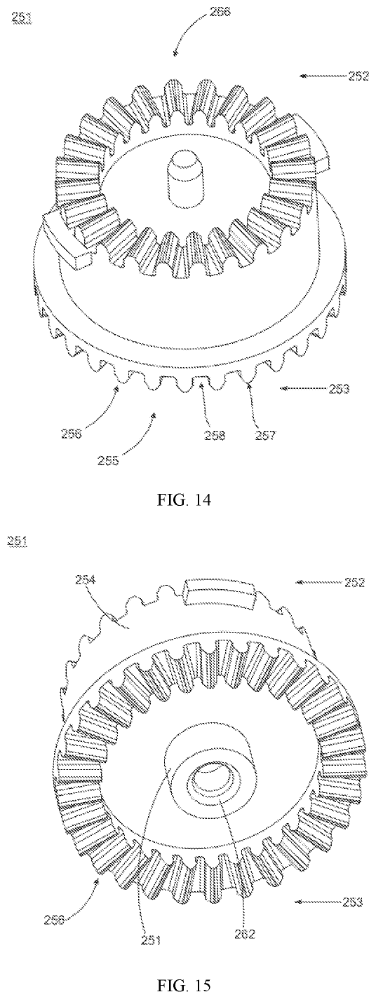

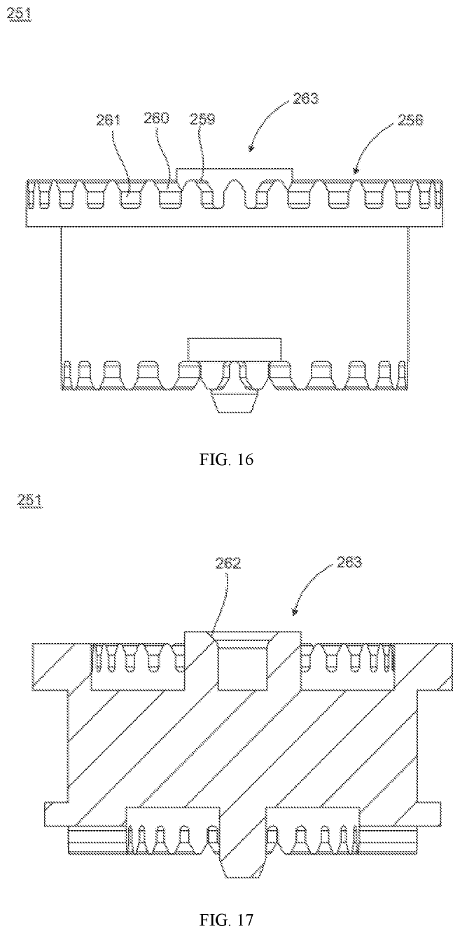

FIG. 14, FIG. 15, FIG. 16, and FIG. 17 illustrate the transmission and connection member 251 for the sterile adapter in the second optional embodiment of the present disclosure.

The transmission and connection member 251 for the sterile adapter has a second drive end 252 and a second connection end 253, which are similar to the second drive end 152 and the second connection end 153 of the transmission and connection member 151 for the sterile adapter 150 in the first optional embodiment and will not be further described here. The transmission and connection member 251 for the sterile adapter further includes a second body portion 254 and a second toothed portion 255 arranged on the edge of the second body portion 254 along a circumferential direction of the second body portion 254. The second toothed portion 255 is arranged at the second connection end 253 and is continuous along circumferential direction. The structure of each of the multiple second teeth 256 may be identical and symmetrical (axial symmetrical).

The second tooth 256 further includes a second tooth peak 257, a second tooth trough 258, a pair of second guide surfaces 259, a pair of second transition surfaces 260, and a pair of second engaging surfaces 261 similar to the second tooth peak 157, the second tooth trough 158, the pair of second guide surfaces 159, the pair of second transition surfaces 160, and the pair of second engaging surfaces 161 in the first optional embodiment. The difference between the second tooth 256 and the second tooth 156 in the first optional embodiment is that there is no matching surfaces on the outer circumference of the second tooth 256. The second tooth 256 is configured to extend along a radial direction of the second body portion 254, and the multiple second teeth 256 are configured to extend over the outer circumference of the second body portion 254. That is, a diameter of the second toothed portion 255 is greater than a diameter of a part of the second body portion 254 close to the second connection end 253.

The transmission and connection member 251 for the sterile adapter further includes a matching portion configured as a positioning cylinder 263 arranged at the second connection end 253 of the second body portion 254 and protrudes from the middle of the second body portion 254. In addition, the multiple second tooth 256 are spaced from the positioning cylinder 263. A second matching inclined surface 262 is formed on a circumference of a top portion of an inner wall of the positioning cylinder 263. The shape of the positioning cylinder 263 matches the shape of the positioning column 223, so that in response to the transmission and connection member 211 for the instrument drive 210 being connected to the transmission and connection member 251 for the sterile adapter, the positioning column 223 extends into the positioning cylinder 263, and the first toothed portion 215 is engaged with the second toothed portion 255.

In addition, a width of the second tooth 256 along a circumferential direction of the second tooth 256 gradually decreases in a radial direction towards the positioning cylinder 263, which can easily cause a partial top surface to be formed at the second tooth peak 257. For example, the outer circumferential side of the second tooth peak 257 shown in FIG. 15 has a top surface, and a sharp corner or a rounded corner is formed at the inner outer circumferential side of the second tooth peak 257. Optionally, the second tooth peak 257 is integrally formed with the sharp corner or the rounded corner.

In the illustrated optional embodiment, a toothed portion may be further arranged on the second drive end 252 of the transmission and connection member 251 for the sterile adapter. For example, the toothed portion is referred to as a third toothed portion 266 (as shown in FIG. 14). The third toothed portion 266 may have the same structure as the second toothed portion 255, except for that third toothed portion 266 has a diameter different from the second toothed portion 255, and will not be further described here. The third toothed portion 266 is configured to mesh with the transmission and connection member for the surgical instrument at the rear end (not shown) of the surgical instrument, in order to further achieve the technical effect of smooth installation in all directions without the need for alignment.

Unless otherwise defined, technical and scientific terms used herein have the same meanings as are commonly understood by those skilled in the art of the present disclosure. Terms used herein are for specific practical purposes only and are not intended to limit the present disclosure. Features described herein in one embodiment may be applied to another embodiment alone or in combination with other features unless the features are not applicable in the other embodiment or otherwise noted.

The present disclosure has been described by way of the above-described embodiments but it shall be understood that the above-described embodiments are for the purpose of illustrative and descriptive only and are not intended to limit the present disclosure to the scope of the described embodiments. Those skilled in the art will appreciate that many more variations and modifications may be made according to the teachings of the present disclosure, all of which fall within the scope of the claims and its equivalents of the present disclosure.

What is claimed is:

1. A transmission and connection structure for connecting an instrument drive and a sterile adapter, wherein the transmission and connection structure comprises:
a first member for transmission connection of the instrument drive, wherein the first member has a first connection end configured to be coupled to the sterile adapter, and the first connection end is provided with a first toothed portion; and
a second member for transmission connection of the sterile adapter, wherein the second member has a second connection end configured to be coupled to the first connection end of the instrument drive, and the second connection end is provided with a second toothed portion;
wherein the second toothed portion is configured to engage with the first toothed portion.

2. The transmission and connection structure according to claim 1, wherein the first toothed portion comprises a plurality of first teeth evenly spaced and arranged along a circumference of the first member, and the second toothed portion comprises a plurality of second teeth evenly spaced and arranged along a circumference of the second member.

3. The transmission and connection structure according to claim 2, wherein the first member further comprises a first body portion, each of the plurality of first teeth is symmetrical about a respective centerline, the second member further comprises a second body portion, and each of the plurality of second teeth is symmetrical about a respective centerline.

4. The transmission and connection structure according to claim 2, wherein each of the plurality of first teeth has a first tooth peak formed as one of a sharp corner and a rounded corner.

5. The transmission and connection structure according to claim 2, each of the plurality of second teeth has a second tooth peak formed as one of a sharp corner and a rounded corner.

6. The transmission and connection structure according to claim 4, wherein each of the plurality of first teeth has two first guide surfaces arranged symmetrically by two sides of the first tooth peak, respectively, and a horizontal distance between the two first guide surfaces gradually increases in a direction away from the first tooth peak, and each of the two first guide surfaces is formed as a flat surface or a smoothly curved surface.

7. The transmission and connection structure according to claim 5, wherein each of the plurality of second teeth has two second guide surfaces arranged symmetrically by two sides of the second tooth peak, respectively, and a horizontal distance between the two second guide surfaces gradually increases in a direction away from the second tooth peak, and each of the two second guide surfaces is formed as a flat surface or a smoothly curved surface.

8. The transmission and connection structure according to claim 2, wherein each of the plurality of first teeth has two first engaging surfaces arranged symmetrically on two sides of a respective first tooth, and each of the two first engaging surfaces is formed as a vertical surface or a slightly inclined surface;
each of the plurality of second teeth has two second engaging surfaces arranged symmetrically on two sides of a respective second tooth, and each of the two second engaging surfaces is formed as a vertical surface or a slightly inclined surface;
in engagement of the first toothed portion with the second toothed portion, each of the two first engaging surfaces is in contact with a corresponding second engaging surface.

9. The transmission and connection structure according to claim 4, wherein each of the plurality of first teeth has a width in a radial cross-section at the first tooth peak smaller than a width in a radial cross-section at a root of a respective first tooth.

10. The transmission and connection structure according to claim 5, wherein each of the plurality of second teeth has a width in a radial cross-section at the second tooth peak smaller than a width in a radial cross-section at a root of a respective second tooth.

11. The transmission and connection structure according to claim 2, wherein each of the plurality of first teeth has a first smooth arc surface at a periphery of the first toothed portion, the first smooth arc surface is a physical surface of a respective first tooth, and the first smooth arc surface has a diameter in a radial cross-section of the first toothed portion, the diameter of the first smooth arc surface gradually increases along a direction from a first tooth peak to a root of a respective first tooth.

12. The transmission and connection structure according to claim 11, wherein the second toothed portion has a plurality of connecting surfaces and each of which is arranged between two respective adjacent second teeth of the second toothed portion, each of the plurality of connecting surfaces connects two respective engaging surfaces each corresponding to one of the respective two adjacent second teeth, and each of the plurality of connecting surfaces is partly formed as a second matching inclined surface, the second matching inclined surface has a diameter in a radial cross-section of the second toothed portion, the diameter of the second matching inclined surface gradually decreases along a direction from a second tooth peak to a root of a respective second tooth, and the second matching inclined surface is configured to slidably fit with the first smooth arc surface at the periphery of the first toothed portion.

13. The transmission and connection structure according to claim 12, wherein each of the first teeth has a first cylindrical surface at a periphery of the first toothed portion, the first cylindrical surface is a physical surface of the respective first tooth, and the first cylindrical surface is adjacent to the first smooth arc surface and extends to the root of each of the plurality of first teeth; and
the second toothed portion has the plurality of connecting surfaces and each of which is arranged between two respective adjacent second teeth of the second toothed portion, and each of the plurality of connecting surfaces is partly formed as a second matching cylindrical surface adjacent to the second matching inclined surface, and the second matching cylindrical surface is configured to fit with the first cylindrical surface of the first toothed portion.

14. The transmission and connection structure according to claim 3, wherein the first member further comprises a positioning portion disposed at the first connection end and arranged at the first body portion, the first toothed portion is arranged around the positioning portion, and the positioning portion comprises a first matching inclined surface extending along a circumference of a top portion of the positioning portion.

15. The transmission and connection structure according to claim 14, wherein the second member comprises a matching portion disposed at the second connection end and arranged correspondingly to the positioning portion, the positioning portion extends at least partially into the matching portion, the second toothed portion is arranged along a circumference of the matching portion, and the matching portion has a second matching inclined surface arranged along the circumference of the matching portion.

16. The transmission and connection structure according to claim 15, wherein the second member further comprises a second drive end opposite to the second connection end, the positioning portion is configured as a protruding portion protruding from the first body portion, the first toothed portion is connected to the protruding portion, the matching portion is configured as a recessing portion, the recessing portion has an opening defined at the second connection end, the recessing portion recesses towards the second drive end, the second toothed portion is arranged on an inner wall of the recessing portion, and the second matching inclined surface is arranged at a circumference, at the second connection end, of the inner wall of the recessing portion.

17. The transmission and connection structure according to claim 15, wherein the positioning portion is configured as a positioning column protruding from the first body portion, the first toothed portion is spaced from the positioning column, the matching portion is configured as a positioning cylinder protruding from the second body portion, the second matching inclined surface is arranged at a circumference of a top portion of an inner wall of the positioning cylinder, the positioning column extends at least partially into the positioning cylinder, and the first matching inclined surface is configured to fit with and guide the second matching inclined surface.

18. The transmission and connection structure according to claim 17, wherein a width of each of the plurality of first teeth along a circumference of a respective first tooth gradually decreases in a radial direction towards the positioning column, and a width of each of the plurality of second teeth along a circumference of a respective second tooth gradually decreases in a radial direction towards the positioning cylinder.

19. A surgical robot, comprising a transmission and connection structure according to claim 1.
